SEQUENCE LISTING (3 sequences)

SEQ ID NO: 1 - protein, 9 aa, synthetic construct
  REGION 1..9: note = The sequence is synthetized.
QGDVVALPA

SEQ ID NO: 2 - protein, 518 aa, synthetic construct
  REGION 1..518: note = The sequence is synthetized.
MATTRFPSLLFYSCIFLLCNGSMAQLFGQSFTPWQSSRQGGLRGCKFDRLQAFEPLRQVRSQAGITEYFDEQNEQFRCAGVSVIRRVIEPQGLLLPQYHNAPGLVYILQGRGFTGLTFPGCPATFQQQFQQFDQARFAQGQSKSQNLKDEHQRVHHIKQGDVVALPAGIVHWCYNDGDAPIVAVYVFDVNNNANQLEPRQKEFLLAGNNKREQQFGQNIFSGFSVQLLSEALGISQQAAQKIQSQNDQRGEIIRVSQGLQFLKPFVSQQGPVEHQAYQPIQSQQEQSTQYQVGQSPQYQEGQSTQYQSGQSWDQSFNGLEENFCSLEARQNIENPKRADTYNPRAGRITHLNSKNFPTLNLVQMSATRVNLYQNAILSPYWNINAHSVMHMIQGRARVQVVNNHGQTVFNDILRRGQLLIIPQHYVVLKKAEREGCQYISFKTTPNSMVSYIAGKTSILRALPVDVLANAYRISRQESQNLKNNRGEEFGAFTPKFAQTGSQSYQDEGESSSTEKASE

SEQ ID NO: 3 - protein, 12 aa, synthetic construct
  REGION 1..12: note = The sequence is synthetized.
DVNNNANQLEPR

(19) United States
(12) Patent Application Publication
(10) Pub. No.: US 2024/0109942 A1
(43) Pub. Date: Apr. 4, 2024

(54) CHOLECYSTOKININ (CCK) SECRETION-PROMOTING PEPTIDE TARGETING CALCIUM-SENSING RECEPTOR, AND PREPARATION METHOD AND USE THEREOF

(71) Applicant: University of Shanghai for Science and Technology, Shanghai (CN)

(72) Inventors: Xiao GUAN, Shanghai (CN); Hongdong SONG, Shanghai (CN); Kai HUANG, Shanghai (CN); Qiuyun FU, Shanghai (CN)

(73) Assignee: University of Shanghai for Science and Technology, Shanghai (CN)

(21) Appl. No.: 18/539,344

(22) Filed: Dec. 14, 2023

Related U.S. Application Data
(63) Continuation of application No. PCT/CN23/91021, filed on Apr. 27, 2023.

(30) Foreign Application Priority Data
Jun. 27, 2022 (CN) 202210735384.6

(51) Int. Cl. C07K 14/415; A61P 3/04

(52) U.S. Cl. CPC C07K 14/415; A61P 3/04

(57) ABSTRACT

A cholecystokinin (CCK) secretion-promoting peptide targeting a calcium-sensing receptor (CaSR), and a preparation method and use thereof are provided. The CCK secretion-promoting peptide targeting a CaSR is a QGDVVALPA active peptide and has an amino acid sequence as follows: Gln-Gly-Asp-Val-Val-Ala-Leu-Pro-Ala. Compared with the prior art, the active peptide is allowed to be artificially synthesized through a chemical solid phase synthesis method, and is allowed to be obtained by an enzymatic hydrolysis of oat protein, separation and purification. The active peptide is allowed to target the CaSR of an intestinal endocrine cell membrane to activate a Gq signal pathway, thereby further increasing an intracellular calcium ion concentration to significantly promote secretion of CCK by an intestinal endocrine cell; and moreover, the active peptide has the advantages of safety, no toxic side effect, tolerance to digestive enzyme hydrolysis of a gastrointestinal tract, easy absorption, etc.

### CROSS REFERENCE TO THE RELATED APPLICATIONS

This application is a continuation application of International Application No. PCT/CN2023/091021, filed on Apr. 27, 2023, which is based upon and claims priority to Chinese Patent Application No. 202210735384.6, filed on Jun. 27, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZYGJ171 Sequence_Listing.xml, created on 11/23/2023, and is 5,222 bytes in size.

### TECHNICAL FIELD

The present invention belongs to the field of bioactive peptides, and particularly relates to a cholecystokinin (CCK) secretion-promoting peptide targeting the calcium-sensing receptor, and a preparation method and use thereof.

### BACKGROUND

Obesity has been emerged as a global public health issue. A wide range of chronic diseases such as cardiovascular diseases, type II diabetes, atherosclerosis, fatty liver and gout are all associated with obesity. Imbalance between energy intake and energy expenditure directly leads to individual overweight and obesity. In view of this, it is optimal to limit excessive energy intake, i.e. limit food intake, in order to prevent obesity. The hypothalamus and digestive tract constitute the key parts of body appetite control, in which a wide range of brain-gut peptides associated with regulation of food intake are distributed. Cholecystokinin (CCK) is a member of a classic brain-gut peptides, which can regulate the body to produce a satiety signal, and reduce food intake of the body, thereby achieving the effect of suppressing appetite. It is of great significance to increase secretion of the intestinal CCK to prevent and alleviate obesity.

Researches have confirmed that the secretion of the CCK is regulated by dietary factors. As a result, dietary regulation of secretion of the intestinal CCK can improve or alleviate obesity and related chronic diseases. Food-derived bioactive peptides are common dietary factors featuring easy digestion and absorption by human body and high food safety. Many countries have made significant effects to promote the development of food-derived bioactive peptide industry, and clearly pointed out that it is necessary to accelerate development of functional food, support development of functional foods such as the bioactive peptide, and carry out related application. At present, the food-derived bioactive peptides are mainly derived from animal and plant proteins. Increasing attentions have been paid to the plant proteins because of environmental, economic, and sustainable considerations. Avena sativa L, a widely cultivated crop worldwide, has important edibleness. Its protein content is higher than that of other crops such as wheat, corn, and rice. At present, oat polysaccharides and oils are widely developed and utilized, but there is a lack of development and utilization of oat proteins. The development of the bioactive peptides by using proteins deriving from Avena sativa L as a raw material has important application value and development prospect.

### SUMMARY

An objective of the present invention is to provide a cholecystokinin (CCK) secretion-promoting peptide targeting calcium-sensing receptor (CaSR), and a preparation method and use thereof.

The objective of the present invention can be implemented by the following technical solution:

In a first aspect of the present invention, a CCK secretion-promoting peptide targeting a CaSR is provided. The CCK secretion-promoting peptide targeting a CaSR is an active peptide of QGDVVALPA, and has an amino acid sequence as follows: Gln-Gly-Asp-Val-Val-Ala-Leu-Pro-Ala shown as SEQ ID NO: 1.

An oat protein is mainly globulin, where avena 12S seed storage globulin 1 has an amino acid sequence shown as SEQ ID NO: 2. It may be found that the active peptide of QGDVVALPA exists in the oat protein. The active peptide of QGDVVALPA may be prepared from the oat protein.

The active peptide of QGDVVALPA according to the present invention is derived from Avena sativa L, and can target the CaSR to activate a Gq signal pathway, thereby further increasing an intracellular calcium ion concentration to significantly promote secretion of CCK in the enteroendocrine cells; and moreover, the active peptide has the advantages of safety, no toxic side effect, tolerance to digestive enzyme hydrolysis of a gastrointestinal tract, easy absorption, etc.

In a second aspect of the present invention, a polynucleotide encoding the CCK secretion-promoting peptide targeting a CaSR is provided.

In a third aspect of the present invention, a preparation method of the CCK secretion-promoting peptide targeting a CaSR is provided. The CCK secretion-promoting peptide targeting a CaSR is artificially synthesized through a genetic engineering method, or is directly obtained from oat protein through a separation and purification method, or is directly prepared through chemical synthesis.

Artificial synthesis of the CCK secretion-promoting peptide targeting a CaSR through the genetic engineering method is a technical solution achievable by those skilled in the art. For example, sequence synthesis of polypeptide can be controlled by means of an appropriate deoxyribonucleic acid (DNA) template on the basis of a recombinant DNA technology.

The manner of directly obtaining the CCK secretion-promoting peptide targeting a CaSR from the oat protein through the separation and purification method may be as follows: on the basis of a given amino acid sequence of the CCK secretion-promoting peptide targeting a CaSR, the CCK secretion-promoting peptide targeting a CaSR is obtained from Avena sativa L through conventional enzymatic hydrolysis, separation and purification methods using a biological technology.

The preparation method through chemical synthesis synthesizes the oligopeptide described above through a traditional solid phase synthesis method.

In a fourth aspect of the present invention, a preparation method of an enzymatic hydrolysate containing the CCK secretion-promoting peptide targeting a CaSR is provided. The preparation method enzymatically hydrolyses oat protein by means of a two-step enzymatic hydrolysis method, and includes: sequentially enzymatically hydrolyzing the oat protein through pepsin and trypsin to obtain an oat protein hydrolysate, i.e. enzymatic hydrolysate containing the CCK secretion-promoting peptide targeting a CaSR.

In an embodiment of the present invention, the sequentially enzymatically hydrolysing the oat protein through pepsin and trypsin includes:
1) extracting the oat protein from Avena sativa L;
2) enzymatically hydrolysing the oat protein through the pepsin to obtain a primary enzymatic hydrolysate;
3) enzymatically hydrolysing the primary enzymatic hydrolysate through the trypsin to obtain an oat protein hydrolysate.

In an embodiment of the present invention, the extracting the oat protein from Avena sativa L includes:
grinding the Avena sativa L into powder, degreasing the powder, then adding distilled water, adjusting pondus Hydrogenii (pH) to 4-6, and pretreating a solution through cellulase for 0.5 h-2 h; then adjusting pH to 10-12 for protein extraction, after completion, centrifuging a solution to obtain supernate, adjusting pH of the supernate to 4.4-4.6, leaving the supernate to stand, centrifuging same, and washing same; and finally drying to obtain the oat protein.

In an embodiment of the present invention, a mass ratio of the pepsin or the trypsin to the oat protein is 1:10-100.

In an embodiment of the present invention, an enzymatic hydrolysis condition of the pepsin or the trypsin is enzymatic hydrolysis for 1 h-4 h at 37° C.

In an embodiment of the present invention, step 3) of the obtaining an oat protein hydrolysate further includes:
separating the oat protein hydrolysate through a Toyopearl HW-40F size exclusion chromatographic column, and eluting and chromatographically purifying a separated oat protein hydrolysate by using deionized water as an eluent at a certain flow rate, to collect different components; and then detecting an influence of different components on secretion of CCK by an intestinal endocrine cell, and selecting a component having the highest activity for stimulating secretion of the CCK as a target component, i.e. the enzymatic hydrolysate containing the CCK secretion-promoting peptide targeting a CaSR.

In a fifth aspect of the present invention, an enzymatic hydrolysate of the CCK secretion-promoting peptide targeting a CaSR prepared on the basis of the preparation method described above is provided.

In a sixth aspect of the present invention, a use of a CCK secretion-promoting peptide targeting a CaSR and an enzymatic hydrolysate containing the CCK secretion-promoting peptide targeting a CaSR in preparation of a product having at least one function of 1)-4) as follows:
1) promoting secretion of CCK;
2) slowing down gastric emptying;
3) suppressing appetite to reduce food intake; and
4) preventing or adjunctively treating obesity.

The CCK secretion-promoting peptide targeting a CaSR and the enzymatic hydrolysate containing the CCK secretion-promoting peptide targeting a CaSR have the function of promoting secretion of CCK, and can slow down gastric emptying, suppress appetite to reduce food intake, and prevent or adjunctively treat obesity.

In a seventh aspect of the present invention, a product is provided. The product includes the CCK secretion-promoting peptide targeting a CaSR and the enzymatic hydrolysate containing the CCK secretion-promoting peptide targeting a CaSR, and has at least one function of 1)-4) as follows:
1) promoting secretion of CCK;
2) slowing down gastric emptying;
3) suppressing appetite to reduce food intake; and
4) preventing or adjunctively treating obesity.

The product includes food, functional food/health food and medicine.

In an eighth aspect of the present invention, a use of the CCK secretion-promoting peptide targeting a CaSR and the enzymatic hydrolysate containing the CCK secretion-promoting peptide targeting a CaSR in preparation of a kit. The kit is configured to activate the CaSR or a downstream Gq signal pathway of the CaSR.

Compared with the prior art, the present invention has the advantages and beneficial effects as follows:
the present invention screens out the active peptide having the function of promoting secretion of intestinal CCK. The active peptide has a novel peptide sequence structure, and no other relevant report has been seen so far; the active peptide of the present invention can be prepared from food protein oat protein and has the advantages of safety and no toxic side effect; the active peptide of QGDVVALPA can tolerate digestion of the pepsin and the pancreatin in a gastrointestinal tract and has excellent stability, thereby maximally exerting CCK secretion-promoting activity; the active peptide of QGDVVALPA of the present invention has molecular weight less than 1000 Da and small molecular weight, can promote the secretion of the intestinal CCK, and is further easy to absorb by a body, thereby having an excellent nutritional function; the preparation method of the QGDVVALPA or the enzymatic hydrolysate containing the sequence peptide of QGDVVALPA according to the present invention is simple and easy to operate, facilitates industrial large-scale production, and has a wide application prospect.

### BRIEF DESCRIPTION OF THE DRAWINGS

FIGS. 1A-1B are a liquid phase diagram and a mass spectrum diagram of an active peptide sequence of QGDVVALPA after artificial synthesis;

FIG. 2 shows the effect of an active peptide sequence of QGDVVALPA on viability of an STC-1 cell;

FIG. 3 shows the effect of an active peptide sequence of QGDVVALPA on secretion of cholecystokinin (CCK) by an STC-1 cell;

FIG. 4 shows the effect of different concentrations of oat protein hydrolysate on secretion of CCK by an STC-1 cell;

FIG. 5 shows the effect of an oat protein hydrolysate on secretion of CCK by an intestinal endocrine cell in a mouse;

FIG. 6 is a chromatogram of an oat protein hydrolysate separated by an HW-40F size exclusion chromatographic column;

FIG. 7 shows the effect of separated components on secretion of CCK by an STC-1 cell;

FIG. 8 is an MS/MS spectrum and sequence analysis of a peptide of QGDVVALPA in the separated component F1; and

FIGS. 9A-9C show the effect of a peptide of QGDVVALPA on secretion of CCK by an STC-1 cell under different inhibitors.

### DETAILED DESCRIPTION OF THE EMBODIMENTS

The present invention will be described in detail below with reference to the accompanying drawings and particular examples.

### Example 1 Artificial Synthesis of an Active Peptide of QGDVVALPA and Evaluation of Cholecystokinin (CCK) Secretion-Promoting Activity

I. Synthesis of the Active Peptide of QGDVVALPA

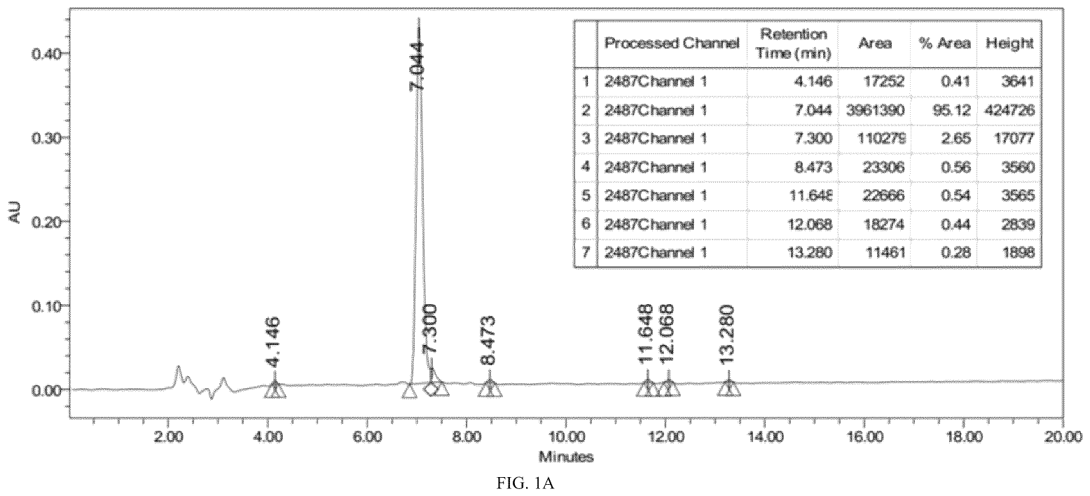

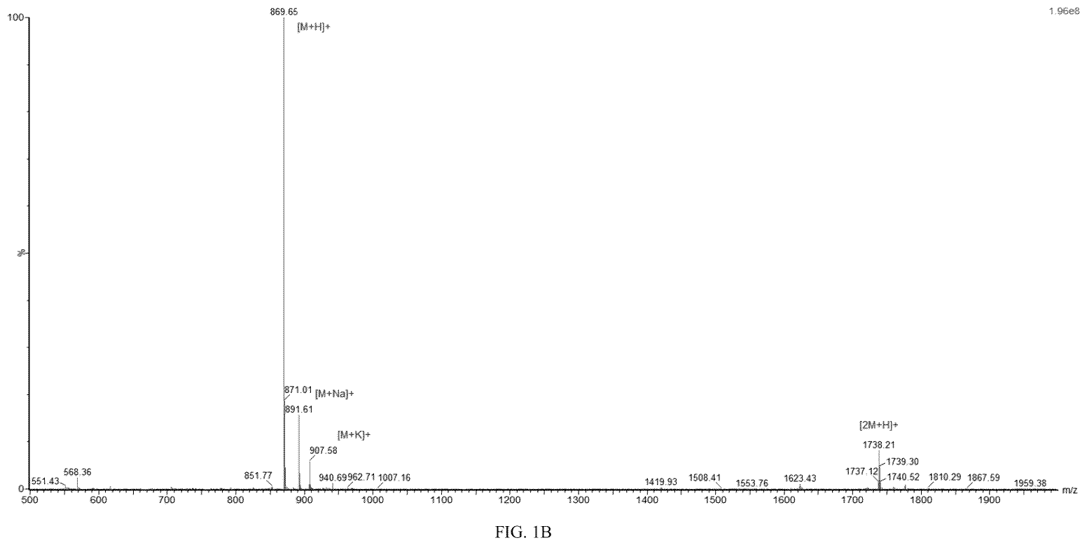

An active peptide of Gln-Gly-Asp-Val-Val-Ala-Leu-Pro-Ala(QGDVVALPA) was synthesized by “Zhejiang Hongtuo Technology Co., Ltd.” through a peptide solid phase synthesis method. Purity of the synthesized peptide was verified to be greater than 95% by means of a high-performance liquid chromatography and a mass spectrometry technology. A liquid phase diagram and a mass spectrum diagram of QGDVVALPA were shown in FIGS. 1A-1B.

II. Effects of QGDVVALPA on Viability and Secretion of CCK of SCT-1 Cell

(1) STC-1 Cell Culture

An STC-1 cell was cultured in a dulbecco's modified eagle medium (DMEM) containing 10% fetal bovine serum (FBS), 1% non-essential amino acids (NEAA), 100 U/mL penicillin and 0.1 mg/mL streptomycin. The cell was incubated in a cell incubator containing 5% CO₂ at 37° C. and was subcultured by means of trypsin digestion when reaching 80%-90% density.

(2) Cell Activity Assay

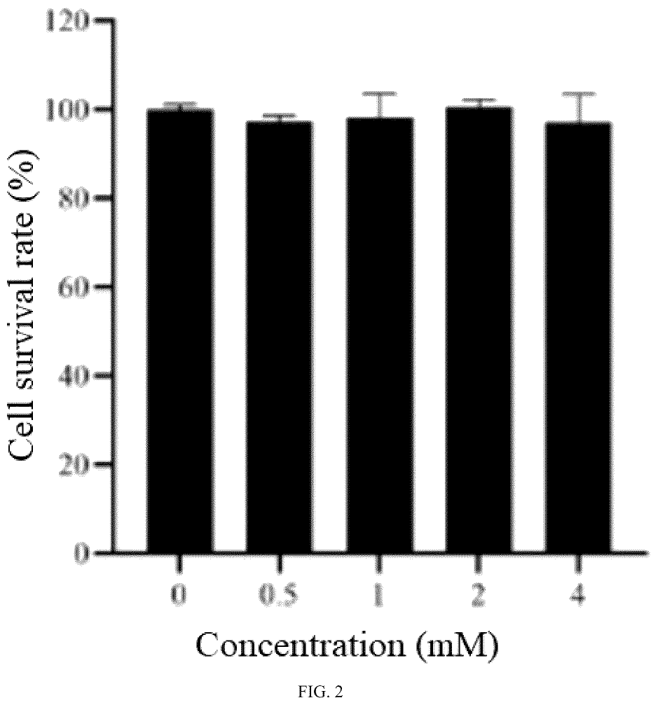

The effect of the active peptide of QGDVVALPA on the viability of the STC-1 cell was tested and evaluated by means of 3-(4,5-dimethylthiazol-2-yl)-2,5-diphenyltetrazolium bromide [3-(4,5-di methyl-2-thiazolyl)-2,5-di phenyl]-2H-tetrazolium bromide, methyl thiazolyl tetrazolium (MTT)) cell proliferation and cytotoxicity. MTT was added into the STC-1 cell treated in a 96-well plate, and a metabolically active cell cleaved yellow tetrazolium salt MTT into a purple formazan crystal. Formazan formed was dissolved, absorbance was measured with a microplate reader at a measurement wavelength of 570 nm, and results were expressed as a percentage of a control group. Results were shown in FIG. 2. The viability of the STC-1 cell was not changed compared with the control group at different peptide concentrations tested (0 mmol/L, 1 mmol/L, 2 mmol/L, 4 mmol/L), which was indicated that the active peptide of QGDVVALPA was non-toxic to the cell.

(3) Assay of Secretory Hormone Content of STC-1 Cell

The active peptide of QGDVVALPA was prepared into peptide solutions having molar concentrations of 0.2 mM, 2 mM and 5 mM respectively with a Hank's buffer solution. The STC-1 cell was inoculated in a 24-well culture plate at a density of 1.25*10⁵ cells. When the cell reached 80%-90% confluence, the cell was washed twice with the Hank's buffer solution to remove the medium. Seven oligopeptide solutions were added into the STC-1 cell, and the cell was incubated in an incubator for 2 h at 37° C.. At the end of incubation, 1000 g of solution was centrifuged for 20 min to obtain supernate. A CCK content was assayed by a commercial CCK kit of Wuhan Cloud Clone Science and Technology Co., Ltd.

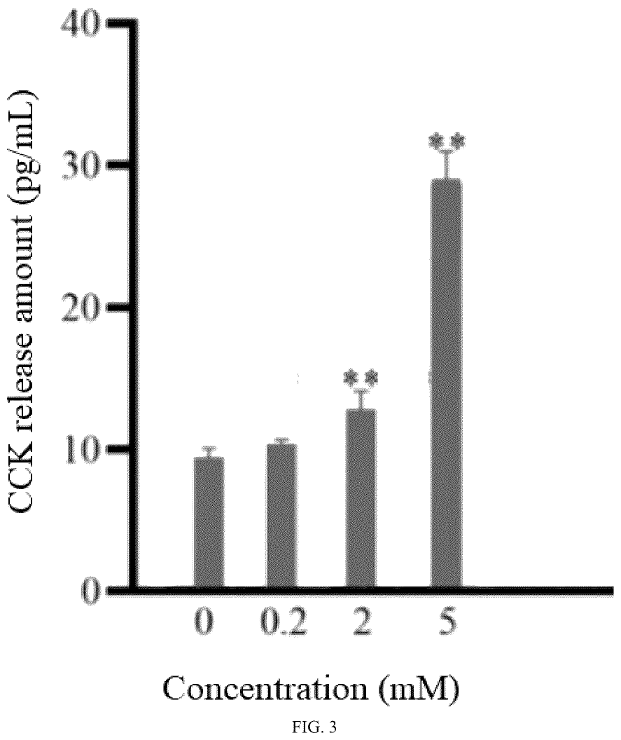

The effect of the active peptide of QGDVVALPA on secretion of CCK by the STC-1 cell was shown in FIG. 3, and it may be seen that the active peptide of QGDVVALPA increased secretion of the CCK in a dose-dependent manner. A large number of studies have confirmed that the CCK was an important member of a brain-gut peptide, which may regulate a body to produce a satiety signal, and reduce food intake of the body, thereby achieving the effect of suppressing appetite. It was of great significance to increase the secretion of the intestinal CCK to prevent and alleviate obesity. The active peptide of QGDVVALPA of the present patent can significantly promote the intestinal endocrine cell STC-1 to secrete the CCK, such that the peptide had important significance and application value for promotion of satiety and loss of weight.

### Example 2 Preparation of Oat Protein Hydrolysate and Evaluation of Activity

(1) Preparation of Oat Protein Hydrolysate

Avena sativa L was ground into powder through an 80 mesh, and then the Avena powder was degreased with hexane. Degreased Avena powder was soaked into a beaker through distilled water in a mass ratio of 12:1, pondus Hydrogenii (pH) was adjusted to 5.0 through 1 mol/L HCl, and a solution was treated through cellulase for 1 h at 50° C. Then, the pH was adjusted to 11.0 through 1 mol/L NaOH, and the solution acted for 2 h in a magnetic stirrer to obtain supernate. The pH of the supernate was adjusted to an isoelectric point (pH 4.5) through 1 mol/L HCl, the supernate was left to stand for 1 h, then centrifuged, washed with water, precipitated to neutrality, redissolved through a small amount of distilled water, and frozen and dried to obtain oat protein, and the oat protein was stored at 4° C. for later use.

One g of lyophilized protein powder was dissolved into 20 mL of K₂HPO₄—KH₂PO₄ phosphate buffer solution (0.1 mol/L) containing 25 mg of freshly prepared pepsin, the pH of the solution was adjusted to 2.0 through HCl (1 mol/L), and the solution was incubated for 2 h at 37° C.. At the end of incubation, the pH of the solution was adjusted to 6.8 through NaOH (1 mol/L), and 50 mg of trypsin was added to continue enzymatic hydrolysis for 2 h. After completion, enzyme was inactivated by a boiling water bath for 8 min, the solution was centrifuged to obtain supernate, and the supernate was frozen and dried to obtain oat protein hydrolysate.

(2)Activity Evaluation

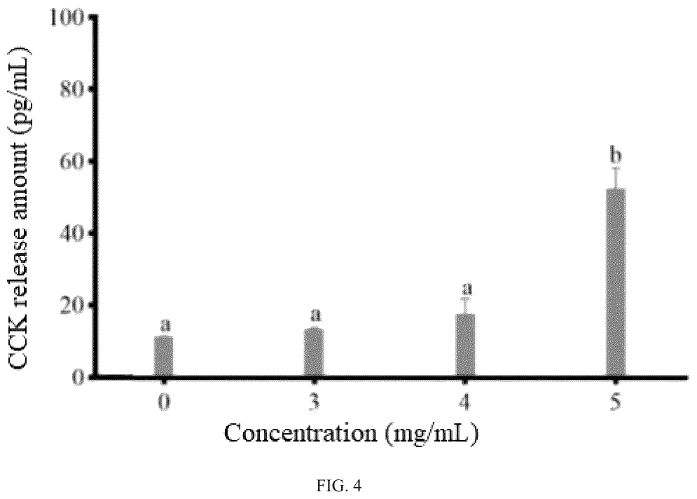

The oat protein hydrolysate was prepared into solutions having mass concentrations of 3 mg/mL, 4 mg/mL and 5 mg/mL separately with a Hank's buffer solution, and the effect of the hydrolysate on secretion of CCK by an STC-1 cell was assayed through the above method. Results were shown in FIG. 4. It may be seen from the results that the oat protein hydrolysate may significantly stimulate secretion of the CCK by the STC-1 cell.

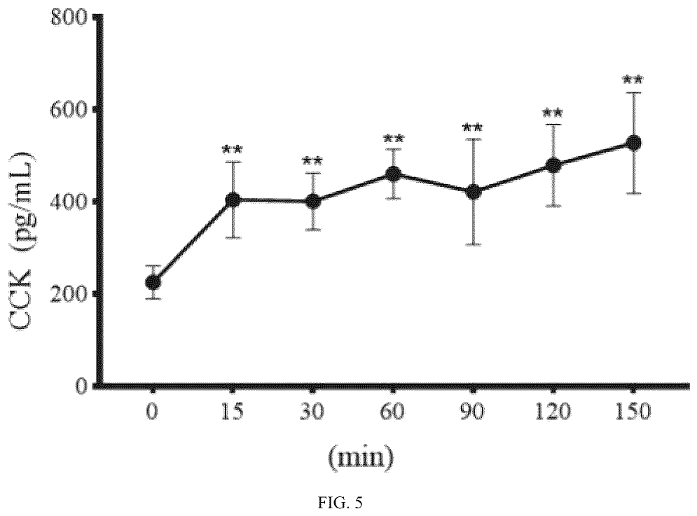

Further, the effect of the oat protein hydrolysate on hormone secretion by the intestinal endocrine cell in a mouse was evaluated at an animal level. After one week of adaptation period, institute for cancer research (ICR) mice were randomly divided into 2 groups (28 mice in each group). Control group: normal saline was administered intragastrically; and oat protein hydrolysate group: oat protein hydrolysate (1.0 g/kg body weight) was administered intragastrically. After intragastric administration, eyeballs were removed at 0 min, 15 min, 30 min, 60 min, 90 min, 120 min and 150 min to collect blood, the blood was put into centrifuge tubes containing ethylene diamine tetraacetic acid (EDTA) (final concentration 1 mg/mL) and aprotinin (final concentration 0.6 TIU/mL), a solution was centrifuged to obtain supernate, and CCK hormone content in serum was assayed through an enzyme-linked immunosorbent assay (ELISA) method. A CCK level in the serum in the intragastric administration normal saline group was maintained at 10 pg/mL during the period. Results of the intragastric administration oat protein hydrolysate group was shown in FIG. 5, and it may be found that the oat protein hydrolysate greatly increased the CCK level in the mouse, and a CCK concentration in blood of the mouse was always about 400 pg/mL within 150 min.

### Example 3 Preparation of CCK Secretion-Promoting Active Peptide of Oat Protein

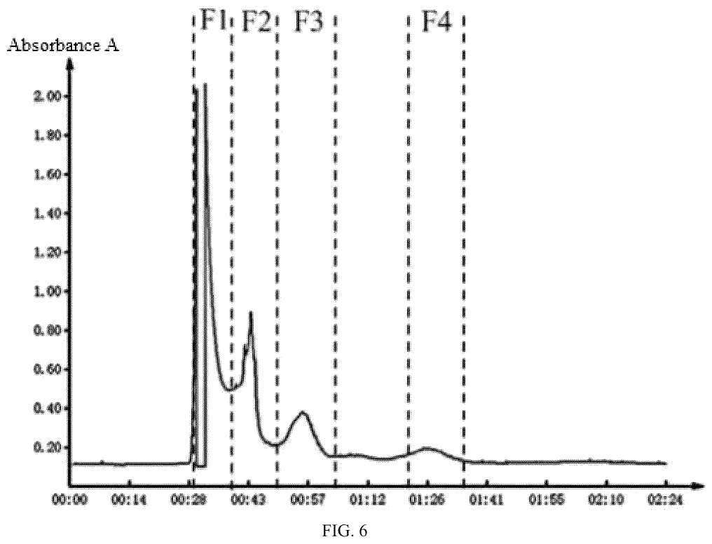

Toyopearl HW-40F packing conventionally swelled and was packed (a packing buffer solution was 0.1 M NaCl dissolved into 50 mM phosphate), a column height was 10 cm, an inner diameter was 2.6 cm, 1.5 cm-2 cm of water layer was needed at a top of a column at any moment, a column may be used after about 3-4 column sizes were balanced, and oat protein hydrolysate was added when 2 mm-3 mm of liquid level was reserved. About 20 mg of oat protein hydrolysate powder was weighed and dissolved into 2 mL of distilled water. Filtration was carried out through a 0.45 μm microporous membrane, then a chromatographic column was added, an eluent was distilled water at an elution speed of 2 mL/min, and an elution peak was collected. A separation graph of the oat protein hydrolysate was shown in FIG. 6. It may be seen that an HW-40F chromatographic column divides the oat protein hydrolysate into 4 peptide components.

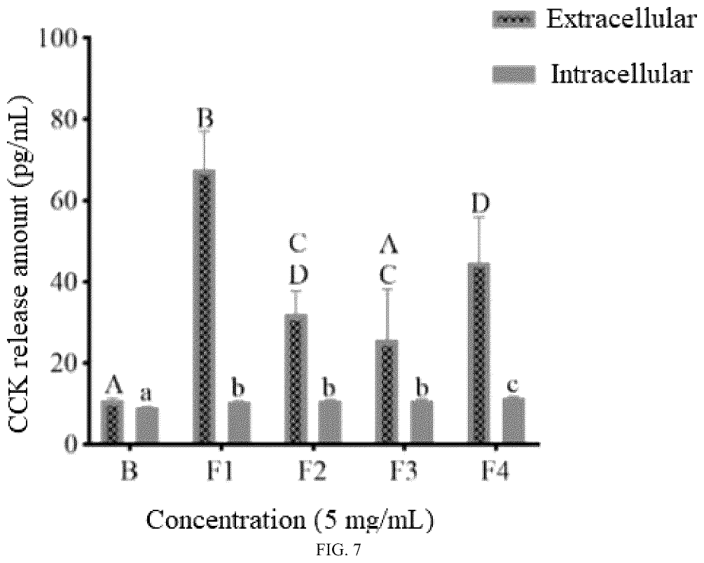

The four peptide components were evaluated for activity through the method described above, and results were shown in FIG. 7. It may be found that in the four peptide components, the component F1 had an optimal capability to stimulate secretion of CCK by an STC-1 cell. Therefore, the component F1 is a CCK secretion-promoting peptide having high activity.

### Example 4 Identification of Oat Protein Containing Active Peptide of QGDVVALPA

A peptide sequence in the component F1 was identified through a mass spectrometry technology. A sample was dissolved into distilled water to prepare a 1 mg/mL sample. Separation was carried out through a reversed phase column (150 μm i.d.*150 mm, packed with Acclaim PepMap RPLC C18, 1.9 μm, 100 Å), a mobile phase A was 0.1% formic acid solution, a mobile phase B was 0.1% formic acid/80% acetonitrile solution, gradient elution was carried out at 600 nL/min of flow rate, and separation gradient was as follows: 0 min-2 min, 4%-8% B; 2 min-45 min, 8%-40% B; 45 min-55 min, 40%-60% B; 55 min-56 min, 60%-95% B; and 56 min-66 min, 95% B. Mass spectrometry had an ion source type of electrospray ionization (ESI), a positive ion scanning mode, a spray voltage of 2200 V, and a capillary temperature of 270° C.. Primary mass spectrometry parameters were set as follows: scanning range of 100 m/z-2000 m/z, maximum resolution of 70000, and automatic gain parameter of 3000000. Secondary mass spectrometry parameters were set as follows: scanning range of 50 m/z-2000 m/z, maximum resolution of 17500, and automatic gain parameter of 100000.

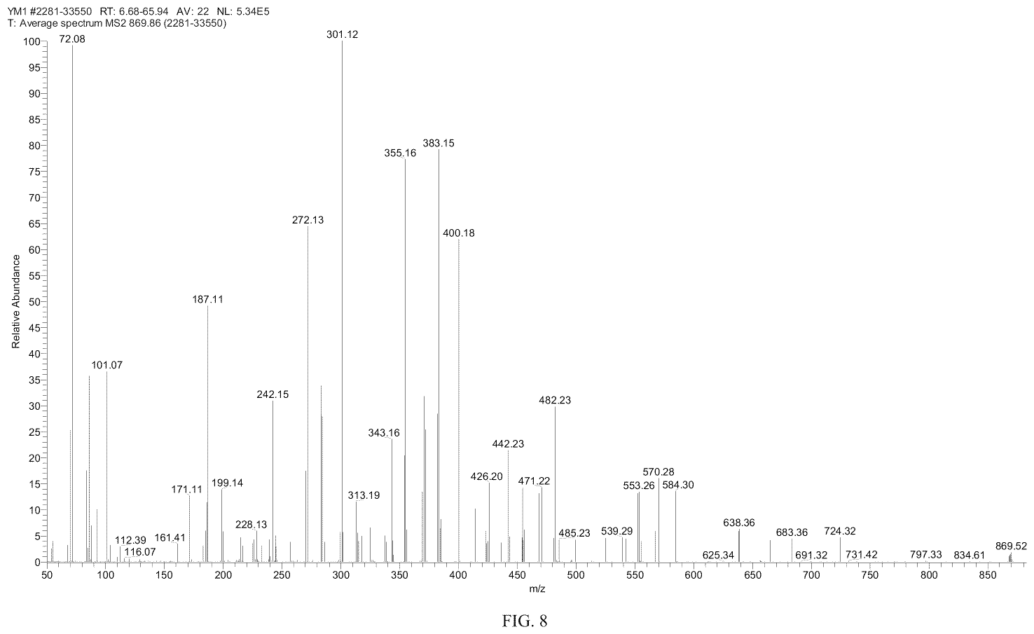

A main ion peak in the component F1 was m/z=869.472 having one charge through mass spectrometry detection. A molecular ion peak was further analyzed by secondary mass spectrometry. A secondary mass spectrum of the molecular ion peak was shown in FIG. 8. A peptide corresponding to the molecular ion peak was Gln-Gly-Asp-Val-Val-Ala-Leu-Pro-Ala (QGDVVALPA) through database matching.

An oat protein was mainly globulin, where avena 12S seed storage globulin 1 had an amino acid sequence shown as SEQ ID NO: 2. It may be found that the active peptide of QGDVVALPA existed in the oat protein. An oligopeptide of DVNNNANQLEPR, as shown in SEQ ID NO: 3, may be prepared from the oat protein.

The amino acid sequence of avena 12S seed storage globulin 1 was specifically as follows:

```
Met Ala Thr Thr Arg Phe Pro Ser Leu Leu Phe Tyr Ser Cys Ile Phe
1 5 10 15
Leu Leu Cys Asn Gly Ser Met Ala Gln Leu Phe Gly Gln Ser Phe Thr
                        20 25 30
Pro Trp Gln Ser Ser Arg Gln Gly Gly Leu Arg Gly Cys Lys Phe Asp
                35 40 45
Arg Leu Gln Ala Phe Glu Pro Leu Arg Gln Val Arg Ser Gln Ala Gly
        50 55 60
Ile Thr Glu Tyr Phe Asp Glu GIn Asn Glu Gln Phe Arg Cys Ala Gly
65 70 75 80
Val Ser Val Ile Arg Arg Val Ile Glu Pro Gln Gly Leu Leu Leu Pro
                                85 90 95
Gln Tyr His Asn Ala Pro Gly Leu Val Tyr Ile Leu Gln Gly Arg Gly
                        100 105 110
Phe Thr Gly Leu Thr Phe Pro Gly Cys Pro Ala Thr Phe Gln Gln Gln
                115 120 125
Phe Gln Gln Phe Asp Gln Ala Arg Phe Ala Gln Gly Gln Ser Lys Ser
        130 135 140
Gln Asn Leu Lys Asp Glu His Gln Arg Val His His Ile Lys Gln Gly
145 150 155 160
Asp Val Val Ala Leu Pro Ala Gly Ile Val His Trp Cys Tyr Asn Asp
                                165 170 175
Gly Asp Ala Pro Ile Val Ala Val Tyr Val Phe Asp Val Asn Asn Asn
                        180 185 190
Ala Asn Gln Leu Glu Pro Arg Gln Lys Glu Phe Leu Leu Ala Gly Asn
                195 200 205
Asn Lys Arg Glu Gln Gln Phe Gly Gln Asn Ile Phe Ser Gly Phe Ser
        210 215 220
Val Gln Leu Leu Ser Glu Ala Leu Gly Ile Ser Gln Gln Ala Ala Gln
225 230 235 240
Lys Ile Gln Ser Gln Asn Asp Gln Arg Gly Glu Ile Ile Arg Val Ser
                                245 250 255
Gln Gly Leu Gln Phe Leu Lys Pro Phe Val Ser Gln Gln Gly Pro Val
                        260 265 270
Glu His Gln Ala Tyr Gln Pro Ile Gln Ser Gln Gln Glu Gln Ser Thr
                275 280 285
Gln Tyr Gln Val Gly Gln Ser Pro Gln Tyr Gln Glu Gly GIn Ser Thr
        290 295 300
Gln Tyr Gln Ser Gly Gln Ser Trp Asp Gln Ser Phe Asn Gly Leu Glu
305 310 315 320
Glu Asn Phe Cys Ser Leu Glu Ala Arg Gln Asn Ile Glu Asn Pro Lys
                                325 330 335
Arg Ala Asp Thr Tyr Asn Pro Arg Ala Gly Arg Ile Thr His Leu Asn
                        340 345 350
Ser Lys Asn Phe Pro Thr Leu Asn Leu Val Gln Met Ser Ala Thr Arg
                355 360 365
Val Asn Leu Tyr Gln Asn Ala Ile Leu Ser Pro Tyr Trp Asn Ile Asn
        370 375 380
Ala His Ser Val Met His Met Ile GIn Gly Arg Ala Arg Val Gln Val
385 390 395 400
Val Asn Asn His Gly Gln Thr Val Phe Asn Asp Ile Leu Arg Arg Gly
                                405 410 415
Gln Leu Leu Ile Ile Pro Gln His Tyr Val Val Leu Lys Lys Ala Glu
                        420 425 430
Arg Glu Gly Cys Gln Tyr Ile Ser Phe Lys Thr Thr Pro Asn Ser Met
                435 440 445
Val Ser Tyr Ile Ala Gly Lys Thr Ser Ile Leu Arg Ala Leu Pro Val
        450 455 460
Asp Val Leu Ala Asn Ala Tyr Arg Ile Ser Arg Gln Glu Ser Gln Asn
465 470 475 480
Leu Lys Asn Asn Arg Gly Glu Glu Phe Gly Ala Phe Thr Pro Lys Phe
                                485 490 495
Ala Gln Thr Gly Ser Gln Ser Tyr Gln Asp Glu Gly Glu Ser Ser Ser
                        500 505 510
Thr Glu Lys Ala Ser Glu
                515
```

### Example 5 Analysis of Mechanism of Action of Active Peptide of QGDVVALPA

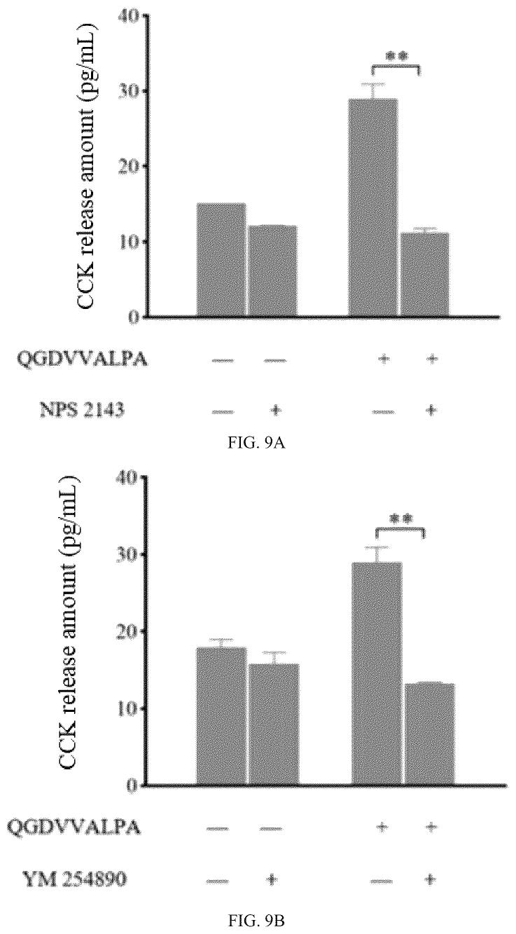

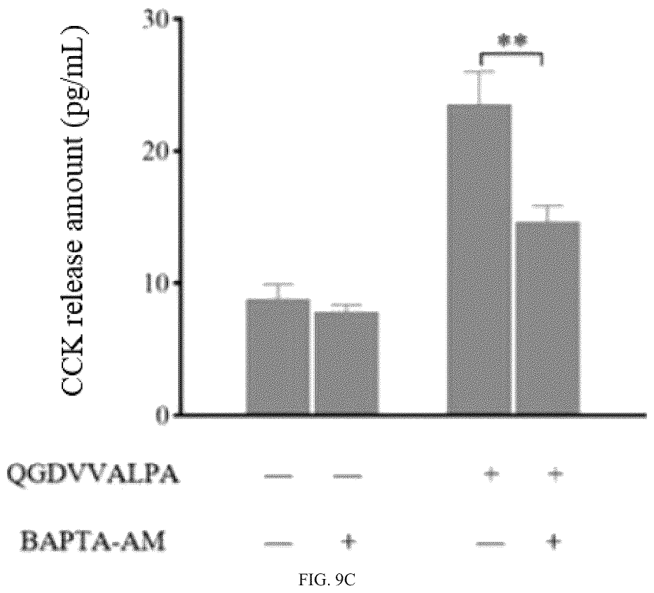

An STC-1 cell was inoculated in a 24-well culture plate at a density of 1.25*10⁵ cells per well. When the cell reached 80%-90% confluence, the cell was washed twice with a Hank's buffer solution to remove a medium, and then pretreated with a calcium-sensing receptor (CaSR) specific antagonist NPS 2143(25 μM), a Gq inhibitor YM 254890 (10 μM), a free calcium ion chelator BAPTA-AM (25 μM) or a carrier (0.1% dimethylsulfoxide (DMSO)) for 10 min separately. The STC-1 cell was exposed to a buffer solution containing an active peptide of QGDVVALPA for 2 h. At the end of incubation, 1000×g of solution was centrifuged for 20 min, and the CCK content outside STC-1 cells was assayed according to operations of description of a cloud clone CCK assay kit. Results were shown in FIGS. 9A-9C. It may be found that a CaSR of a cell membrane mediated a process of the active peptide of QGDVVALPA inducing secretion of CCK by STC-1 cells. Moreover, the secretion of the CCK induced by QGDVVALPA to the STC-1 cell involved activation of a Gq signal pathway and required intracellular Ca²⁺ mobilization.

The above description of the examples is provided to facilitate understanding and use of the invention by those of ordinary skill in the art. Obviously, those skilled in the art can easily make various modifications to these examples and apply the general principles explained herein to other examples without creative efforts. Therefore, the present invention is not limited to the above examples. Improvements and modifications made by those skilled in the art according to the disclosure of the present invention without departing from the scope of the present invention should fall within the scope of protection of the present invention.

### SEQUENCE LISTING

<110>University of Shanghai for Science and Technology
<120>CCK secretion-promoting peptide targeting calcium-sensing receptor, and
preparation method and use thereof
<160>2
<170>SIPOSequenceListing 1.0
<210>1
<211>9
<212>PRT
<213>Avena longiglumis
<400>1

Gln Gly Asp Val Val Ala Leu Pro Ala
1 5

<210>2
<211>518
<212>PRT
<213>Avena longiglumis
<400>2

```
Met Ala Thr Thr Arg Phe Pro Ser Leu Leu Phe Tyr Ser Cys Ile Phe
1 5 10 15
Leu Leu Cys Asn Gly Ser Met Ala Gln Leu Phe Gly Gln Ser Phe Thr
                        20 25 30
Pro Trp Gln Ser Ser Arg Gln Gly Gly Leu Arg Gly Cys Lys Phe Asp
                35 40 45
Arg Leu Gln Ala Phe Glu Pro Leu Arg Gln Val Arg Ser Gln Ala Gly
        50 55 60
Ile Thr Glu Tyr Phe Asp Glu Gln Asn Glu Gln Phe Arg Cys Ala Gly
65 70 75 80
Val Ser Val Ile Arg Arg Val Ile Glu Pro Gln Gly Leu Leu Leu Pro
                                85 90 95
Gln Tyr His Asn Ala Pro Gly Leu Val Tyr Ile Leu Gln Gly Arg Gly
                        100 105 110
Phe Thr Gly Leu Thr Phe Pro Gly Cys Pro Ala Thr Phe Gln Gln Gln
                115 120 125
Phe Gln Gln Phe Asp Gln Ala Arg Phe Ala Gln Gly Gln Ser Lys Ser
        130 135 140
Gln Asn Leu Lys Asp Glu His Gln Arg Val His His Ile Lys Gln Gly
145 150 155 160
Asp Val Val Ala Leu Pro Ala Gly Ile Val His Trp Cys Tyr Asn Asp
                                165 170 175
Gly Asp Ala Pro Ile Val Ala Val Tyr Val Phe Asp Val Asn Asn Asn
                        180 185 190
Ala Asn Gln Leu Glu Pro Arg Gln Lys Glu Phe Leu Leu Ala Gly Asn
                195 200 205
Asn Lys Arg Glu Gln Gln Phe Gly Gln Asn Ile Phe Ser Gly Phe Ser
        210 215 220
Val Gln Leu Leu Ser Glu Ala Leu Gly Ile Ser Gln Gln Ala Ala Gln
225 230 235 240
Lys Ile Gln Ser Gln Asn Asp Gln Arg Gly Glu Ile Ile Arg Val Ser
                                245 250 255
Gln Gly Leu Gln Phe Leu Lys Pro Phe Val Ser Gln Gln Gly Pro Val
                        260 265 270
Glu His Gln Ala Tyr Gln Pro Ile Gln Ser Gln Gln Glu Gln Ser Thr
                275 280 285
Gln Tyr Gln Val Gly Gln Ser Pro Gln Tyr Gln Glu Gly Gln Ser Thr
        290 295 300
Gln Tyr Gln Ser Gly Gln Ser Trp Asp Gln Ser Phe Asn Gly Leu Glu
305 310 315 320
Glu Asn Phe Cys Ser Leu Glu Ala Arg Gln Asn Ile Glu Asn Pro Lys
                                325 330 335
Arg Ala Asp Thr Tyr Asn Pro Arg Ala Gly Arg Ile Thr His Leu Asn
                        340 345 350
Ser Lys Asn Phe Pro Thr Leu Asn Leu Val Gln Met Ser Ala Thr Arg
                355 360 365
Val Asn Leu Tyr Gln Asn Ala Ile Leu Ser Pro Tyr Trp Asn Ile Asn
        370 375 380
Ala His Ser Val Met His Met Ile Gln Gly Arg Ala Arg Val Gln Val
385 390 395 400
Val Asn Asn His Gly Gln Thr Val Phe Asn Asp Ile Leu Arg Arg Gly
                                405 410 415
Gln Leu Leu Ile Ile Pro Gln His Tyr Val Val Leu Lys Lys Ala Glu
                        420 425 430
Arg Glu Gly Cys Gln Tyr Ile Ser Phe Lys Thr Thr Pro Asn Ser Met
                435 440 445
Val Ser Tyr Ile Ala Gly Lys Thr Ser Ile Leu Arg Ala Leu Pro Val
        450 455 460
Asp Val Leu Ala Asn Ala Tyr Arg Ile Ser Arg Gln Glu Ser Gln Asn
465 470 475 480
Leu Lys Asn Asn Arg Gly Glu Glu Phe Gly Ala Phe Thr Pro Lys Phe
                                485 490 495
Ala Gln Thr Gly Ser Gln Ser Tyr Gln Asp Glu Gly Glu Ser Ser Ser
                        500 505 510
Thr Glu Lys Ala Ser Glu
                515
```

What is claimed is:

1. A method of preparing a product, comprising using a cholecystokinin (CCK) secretion-promoting peptide targeting a calcium-sensing receptor and an enzymatic hydrolysate containing the CCK secretion-promoting peptide targeting the calcium-sensing receptor;
wherein the CCK secretion-promoting peptide targeting the calcium-sensing receptor comprises an active peptide of QGDVVALPA, and the active peptide of QGDVVALPA comprises the amino acid sequence as follows: Gln-Gly-Asp-Val-Val-Ala-Leu-Pro-Ala, as shown in SEQ ID NO: 1; and
the product comprises at least one function of promoting a secretion of CCK;
2) slowing down gastric emptying; suppressing an appetite to reduce a food intake, and preventing or adjunctively treating an obesity.
